(19) United States
(12) Patent Application Publication
(10) Pub. No.: US 2007/0281040 A1
(43) Pub. Date: Dec. 6, 2007

(54) COMBINATION THERAPY OF HEDGEHOG INHIBITORS, RADIATION AND CHEMOTHERAPEUTIC AGENTS

(75) Inventors: Ralph R. Weichselbaum, Chicago, IL (US); Zahra Shafaee, Bronxvilles, NY (US); Wei Du, Chicago, IL (US)

Correspondence Address: MICHAEL BEST & FRIEDRICH LLP, ONE SOUTH PINCKNEY STREET, P O BOX 1806, MADISON, WI, 53701, US

(73) Assignee: THE UNIVERSITY OF CHICAGO, Chicago, IL (US)

(21) Appl. No.: 11/576,310

(22) PCT Filed: Sep. 30, 2005
(86) PCT No.: PCT/US05/35331

§ 371 Date: Mar. 29, 2007

Related U.S. Application Data
(60) Provisional application No. 60/614,617, filed on Sep. 30, 2004.
(60) Provisional application No. 60/675,207, filed on Apr. 27, 2005.

(51) Int. Cl. A61K 31/4355; A61K 31/337; A61K 31/56; A61K 31/70; A61K 33/24; A61N 5/10; A61P 35/00

(52) U.S. Cl. 424/649; 514/169; 514/171; 514/278; 514/449; 514/49; 514/789; 600/1

(57) ABSTRACT

The present invention relates to therapeutic combinations and methods of inhibiting the proliferation of cancerous cells, the abnormal growth of cells, and tumor cell growth using the combination of a hedgehog inhibitor with chemotherapy and/or radiation therapy. The present invention also relates to methods of enhancing the antiproliferative effect of chemotherapy and/or radiation therapy in a mammalian cancer patient undergoing either chemotherapy or radiation or a combination of radiation and chemotherapy by co-administering a therapeutically amount of a hedgehog inhibitor, concurrently or sequentially, with the chemotherapy and/or radiation therapy.

### CROSS-REFERENCE TO RELATED APPLICATIONS

This application claims the benefit of U.S. Provisional Application No. 60/614,617, filed Sep. 30, 2004, and U.S. Provisional Application No. 60/675,207, filed Apr. 27, 2005, in their entireties, both of which are hereby incorporated by reference.

### STATEMENT REGARDING FEDERALLY SPONSORED RESEARCH OR DEVELOPMENT

Not Applicable

### BACKGROUND OF THE INVENTION

Therapy for cancer has largely involved the use of chemotherapy, in which highly toxic chemicals are given to the patient, and/or radiotherapy, in which toxic doses of radiation are directed at the patient. Radiation therapy is an established cancer treatment employed in approximately 60% of patients diagnosed with cancer. Radiation therapy is an effective modality when employed alone against very small tumors. For large or radio-resistant tumors, radiotherapy is combined with chemotherapy or hormonal therapy. However, there are many tumors in which radiotherapy even in combination with other treatments fails to achieve tumor cures. For example, radiotherapy combined with chemotherapy is the current treatment for locally advanced pancreatic cancer; however, the results are unsatisfactory with median survivals ranging from 6-10 months. (Klinkenbijl J H, et al., “Adjuvant radiotherapy and 5-fluorouracil after curative resection of cancer of the pancreas and peri-ampullary region. Phase III trial of the EORTC Gastrointestinal Tract Cancer Cooperative Group.” Ann. Surg. 1999;230(6):776-784; and Gastrointestinal Tumor Study Group.)

Similarly, chemotherapeutics that have been used successfully to combat certain cancers are frequently ineffective against other cancers, or are effective only at doses that are so high as to cause unacceptable toxicity. Although cancer chemotherapy has advanced dramatically in recent years, treating cancers with a single agent has had limited success. Further, very few therapeutic agents, including the new “targeted agents” such as EGFR or angiogenesis inhibitors, are curative in human cancer treatment when delivered alone. First, any single agent may only target a subset of the total population of malignant cells present, leaving a subpopulation of cancerous cells to continue growing. Second, cells develop resistance upon prolonged exposure to a drug. Most chemotherapeutic agents are delivered in combination when cures are achieved.

Combination therapies, which employ two or more agents with differing mechanisms of action and differing toxicities, have been useful for circumventing drug resistance and increasing the target cell population. In addition, certain combinations of agents may be synergistic: their combined effect is greater than predicted based on their individual activities. Thus, combining different agents can be a powerful strategy for treating cancer. However, combination therapies are hit or miss. In many cases, cross effects and treatment load and even antagonistic effects can result in lower effectiveness for the combination than either treatment alone. Multidrug resistance can also be a problem.

Thus, a new treatment regimen that can improve the therapeutic ratio for ionizing radiation and/or chemotherapeutic agents is needed for improved, more effective cancer treatment.

### SUMMARY OF THE INVENTION

The present invention provides methods of treating and preventing hyperproliferative diseases, especially cancers, by combining a hedgehog inhibitor with chemotherapy and/or radiation therapy. That is, hedgehog inhibitors may potentiate tumor response to radiation, to chemotherapy, or to a combined treatment of radiation and chemotherapy. Thus, hedgehog inhibitors may improve the efficacy of radiotherapy and/or chemotherapy.

In one embodiment, the present invention provides methods of inhibiting the proliferation of cancerous cells comprising contacting the cells, either concurrently or sequentially, with effective doses of a hedgehog inhibitor, e.g., a steroid alkaloid such as cyclopamine, and a chemotherapeutic agent, e.g., an antimicrotubule agent such as taxol. In another embodiment of the invention, the hedgehog inhibitor and the chemotherapeutic agent are co-administered to a cancer patient (e.g., human or other mammal).

In yet another embodiment of the invention, there is provided a method of enhancing the antiproliferative effect of chemotherapy in a patient with a disease in need of treatment with a chemotherapeutic agent, comprising co-administering to the patient a hedgehog inhibitor and a chemotherapeutic agent.

In a further embodiment, the present invention provides methods of inhibiting the proliferation of cancerous cells comprising contacting the cells, either concurrently or sequentially, with effective doses of a hedgehog inhibitor, e.g., a steroid alkaloid such as cyclopamine, and radiation, e.g., x-radiation or gamma radiation. In another embodiment of the invention, the hedgehog inhibitor and the radiation are co-administered to a cancer patient (e.g., human or other mammal).

In another embodiment, the present invention provides methods of inhibiting the proliferation of cancerous cells comprising contacting the cells, either concurrently or sequentially, with effective doses of a hedgehog inhibitor, radiation, and a chemotherapeutic agent. In another embodiment of the invention, the hedgehog inhibitor and the radiation and chemotherapeutic agent are co-administered to a cancer patient (e.g., human or other mammal).

The methods of the present invention are particularly useful for the treatment or prevention of various cancers, especially epithelial cancers, e.g., prostate cancer, lung cancer, breast cancer, colorectal cancer and pancreatic cancer.

Other advantages and a fuller appreciation of specific adaptations, compositional variations, and physical attributes of the invention will be gained upon an examination of the following detailed description of exemplary embodiments, taken in conjunction with the figures of the drawing. It is expressly understood that the drawings herein are for the purpose of illustration and description only, and are not intended as a definition of the limits of the invention.

### BRIEF DESCRIPTION OF THE DRAWINGS

FIG. 1(A) contains a graph showing the normalized surviving ratio in two pancreatic cell lines (Mia PaCa-2 and BxPC-3) and one colon cancer cell line (HCT 116) following exposure to 4 μMol of cyclopamine, 3.5 Gy of radiation, or a combination of both.

FIG. 1(B) demonstrates the effect on pancreatic and colon cancer cell lines colony formation following exposure to 10 μMol cyclopamine in culture media.

FIG. 2 shows colony formation following exposure to cyclopamine, taxol (3.5 nM), cisplatin (0.8 μMol), and gemcitabine (7.3 nM).

FIG. 3 illustrates the percentage of apoptotic cells following exposure to cyclopamine (4 μMol), taxol (1.7 nMol), or a combination of both for 24 hours (Annexin Assay).

FIG. 4 illustrates the percentage of apoptotic cells following exposure to cyclopamine (4 μMol), radiation (3.5 Gy), or a combination of both for 24, 48, or 72 hours (Annexin Assay).

### DETAILED DESCRIPTION OF THE INVENTION

The present invention includes methods of treating neoplastic or malignant diseases, suitably, those diseases in which the malignant cells express the hedgehog signaling pathway. The methods include use of a hedgehog inhibitor with other anticancer agents, i.e., chemotherapeutic agents or radiation or both, to inhibit abnormal cell growth.

The hedgehog (Hh) signaling pathway plays important roles in tissue growth and organ formation during animal development and in adult tissue homeostasis. Activation of Hh signaling is associated with nonnal tissue repair; however, inappropriate activation of Hh signaling is associated with cancers. Importantly, inhibitors of Hh signaling can inhibit the growth of cancers with deregulated Hh signaling, suggesting that inhibition of Hh signaling is a promising approach to cancer treatment.

The hedgehog signaling pathway is important in tissue growth and differentiation and plays an important role in embryogenesis as well as adult tissue homeostasis. Hedgehog protein gradients are essential for ventral/dorsal patterning in vertebrate central nervous systems and normal development in a variety of tissues including integument, musculoskeletal, gastrointestinal, and urogenital systems, among others. Secreted Hh protein binds the Patched (Ptc) receptor, thereby inhibiting the transmembrane receptor protein Smoothened (Smo). These events allow Hh pathway activation via the downstream transcription factor Gli following nuclear translocation. Activation of Hh signaling has been demonstrated in pancreatic cancer through the overexpression of pathway elements Hh, Ptc, and Gli. For example, the hedgehog signaling pathway is overexpressed in many pancreatic adenocarcinomas. Thayer et al. reported a transgenic model of early pancreatic cancer where Hh overexpression is accompanied by K-ras and Her-2/neu mutations in pancreatic intraepithelial neoplasia, ultimately progressing to invasive adenocarcinoma. (See Thayer, et al., “Hedgehog is an early and late mediator of pancreatic cancer tumorigenesis,” Nature, 425, 851-856 (2003).) Aberrant Hh signaling has also been described in breast, esophagus, gastric, and prostate cancer.

Before any embodiments of the invention are explained in detail, it is understood that all of the compositions and methods disclosed and claimed herein can be made and executed without undue experimentation in light of the present disclosure. While the compositions and methods of this invention have been described in terms of exemplary embodiments, it will be apparent to those skilled in the art that variations may be applied to the compositions and methods and in the steps or in the sequence of steps of the methods described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention.

All patents and publications listed or described herein are incorporated in their entirety by reference.

Unless defined otherwise, all technical and scientific terms used herein have the same meaning as commonly understood by one of ordinary skill in the art to which this invention belongs. For purposes of clarity and as an aid in the understanding of the invention, as disclosed and claimed herein, the following definitions may be useful:

As used herein, “abnormal growth of cells” is meant to refer to cell growth independent of normal regulatory mechanisms (e.g., loss of contact inhibition), including the abnormal growth of benign and malignant cells or other hyperproliferative diseases.

The term “acylamino” is art-recognized and refers to a moiety that can be represented by the general formula: 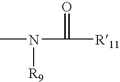
wherein R₉ is as defined above, and R′₁₁ represents a hydrogen, an alkyl, an alkenyl or —(CH₂)ₘ—R₈, where m and R₈ are as defined above.

As used herein, the term “aliphatic group” refers to a straight-chain, branched-chain, or cyclic aliphatic hydrocarbon group and includes saturated and unsaturated aliphatic groups, such as an alkyl group, an alkenyl group, and an alkynyl group.

As used herein, the terms “alkenyl” and “alkynyl” refer to unsaturated aliphatic groups analogous in length and possible substitution to the alkyls described above, but that contain at least one double or triple bond, respectively.

As used herein, the terms “alkoxyl” or “alkoxy” refer to groups of 1 to 8 carbon atoms (C₁-C₈) of a straight, branched, cyclic configuration, and combinations thereof, attached to the parent structure through an oxygen. Examples include methoxy, ethoxy, propoxy, isopropyloxy, tert-butoxy, cyclopropyloxy, cyclohexyloxy, and the like. “Alkoxyl” or “alkoxy” also refers to an alkyl group, as defined above, having an oxygen radical attached thereto. An “ether” is two hydrocarbons covalently linked by an oxygen. Accordingly, the substituent of an alkyl that renders that alkyl an ether is or resembles an alkoxyl, such as can be represented by one of ═O-alkyl, ═O-alkenyl, ═O-alkynyl, ═O—(CH₂)ₘ—R₈, where m and R₈ are described herein.

The term “alkyl” as used herein refers to a radical of saturated aliphatic groups, including straight-chain alkyl groups, branched-chain alkyl groups, cycloalkyl (alicyclic) groups, alkyl-substituted cycloalkyl groups, and cycloalkyl-substituted alkyl groups. In some embodiments, a straight chain or branched chain alkyl has 30 or fewer carbon atoms in its backbone (e.g., C₁-C₃₀ for straight chains, C₃-C₃₀ for branched chains), and more preferably 20 or fewer. Likewise, preferred cycloalkyls have from 3-10 carbon atoms in their ring structure, and more preferably have 5, 6 or 7 carbons in the ring structure. Examples of alkyl groups include methyl, ethyl, 1-propyl, 2-propyl, cyclohexyl, methylcyclopropyl, and the like.

Moreover, the term “alkyl” (or “lower alkyl”) as used throughout the specification, examples, and claims is intended to include both “unsubstituted alkyls” and “substituted alkyls,” the latter of which refers to alkyl moieties having substituents replacing a hydrogen on one or more carbons of the hydrocarbon backbone. Such substituents can include, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain can themselves be substituted, if appropriate. For instance, the substituents of a substituted alkyl may include substituted and unsubstituted forms of amino, azido, imino, amido, phosphoryl (including phosphonate and phosphinate), sulfonyl (including sulfate, sulfonamido, sulfamoyl and sulfonate), and silyl groups, as well as ethers, alkylthios, carbonyls (including ketones, aldehydes, carboxylates, and esters), —CF₃, —CN and the like. Cycloalkyls can be further substituted with alkyls, alkenyls, alkoxys, alkylthios, aminoalkyls, carbonyl-substituted alkyls, —CF₃, —CN, and the like.

Unless the number of carbons is otherwise specified, “lower alkyl” as used herein means an alkyl group, as defined above, but having from one to ten carbons, more preferably from one to six carbon atoms in its backbone structure. Likewise, “lower alkenyl” and “lower alkynyl” have similar chain lengths. Throughout the application, preferred alkyl groups are lower alkyls. In preferred embodiments, a substituent designated herein as alkyl is a lower alkyl.

The term “alkylthio” refers to an alkyl group, as defined above, having a sulfur radical attached thereto. In preferred embodiments, the “alkylthio” moiety is represented by one of —S-alkyl, —S-alkenyl, —S-alkynyl, and —S—(CH₂)ₘ—R₈, wherein m and R₈ are defined above. Representative alkylthio groups include methylthio, ethylthio, and the like.

The terms “amine” and “amino” refer to both unsubstituted and substituted amines, e.g., a moiety that can be represented by the general formula: 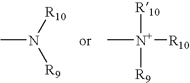
wherein R₉, R₁₀ and R′₁₀ each independently represent a hydrogen, an alkyl, an alkenyl, (CH₂)ₘ—R₈, or R₉ and R₁₀ taken together with the N atom to which they are attached complete a heterocycle having from 4 to 8 atoms in the ring structure; R₈ represents an aryl, a cycloalkyl, a cycloalkenyl, a heterocycle or a polycycle; and m is zero or an integer in the range of 1 to 8. In preferred embodiments, only one of R₉ or R₁₀ can be a carbonyl, e.g., R₉, R₁₀ and the nitrogen together do not form an imide. In even more preferred embodiments, R₉ and R₁₀ (and optionally R′₁₀) each independently represent a hydrogen, an alkyl, an alkenyl, or —(CH₂)ₘ—R₈. Thus, the term “alkylamine” as used herein means an amine group, as defined above, having a substituted or unsubstituted alkyl attached thereto, i.e., at least one of R₉ and R₁₀ is an alkyl group.

The term “amido” refers to an amino-substituted carbonyl and includes a moiety that can be represented by the general formula:

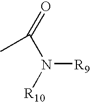

wherein R₉, R₁₀ are as defined above. Preferred embodiments of the amide will not include imides which may be unstable.

The term “aralkyl,” as used herein, refers to an alkyl group substituted with an aryl group (e.g., an aromatic or heteroaromatic group).

“Alkynyl,” as used herein, refers to a linear monovalent hydrocarbon radical of two to six carbon atoms or a branched monovalent hydrocarbon radical of three to six carbon atoms, containing at least one triple bond, e.g., ethynyl, propynyl, and the like.

As used herein, the term “aryl” includes 5-, 6-, and 7-membered single-ring aromatic groups that may include from zero to four heteroatoms, for example, benzene, pyrrole, furan, thiophene, imidazole, oxazole, thiazole, triazole, pyrazole, pyridine, pyrazine, pyridazine and pyrimidine, and the like. Those aryl groups having heteroatoms in the ring structure may also be referred to as “aryl heterocycles” or “heteroaromatics.” The aromatic ring can be substituted at one or more ring positions with such substituents as described above, for example, halogen, azide, alkyl, aralkyl, alkenyl, alkynyl, cycloalkyl, hydroxyl, alkoxyl, amino, nitro, sulfhydryl, imino, amido, phosphate, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF₃, —CN, or the like. The term “aryl” also includes polycyclic ring systems having two or more cyclic rings in which two or more carbons are common to two adjoining rings (the rings are “fused rings”) wherein at least one of the rings is aromatic, e.g., the other cyclic rings can be cycloalkyls, cycloalkenyls, cycloalkynyls, aryls and/or heterocyclyls. Examples of aryl groups include phenyl, naphthyl, and biphenyl.

As used herein, the term “antimicrotubule agent” refers to an agent which interferes with cell division by disrupting the normal functionality of the cellular microtubules. Exemplary antimicrotubule agents may include, but are not limited to, taxanes, such as taxol and taxotere, and vinca alkaloids, such as vincristine and vinblastine.

As used herein, the term “alkylating agent” refers to an agent which generally exerts cytotoxic activity by alkylating DNA, thus directly interfering with the reproductive cycle of the cell. Exemplary alkylating agents may include, but are not limited to, cyclophosphamide, isosfamide, melphalan, hexamethylmelamine, thiotepa or dacarbazine.

As used herein, the term “antimetabolite” refers to an antineoplastic drug that inhibits the utilization of a metabolite and exerts cytotoxic activity by substituting fraudulent nucleotides into cellular DNA, thereby interrupting cell division or inhibiting enzymes which are necessary for DNA replication. Exemplary antimetabolites may include, but are not limited to, pyrimidine analogues, such as 5-fluorouracil, cytarabine, capecitabine, and gemcitabine or its analogues, such as 2-fluorodeoxycytidine; folic acid analogues such as methotrexate, idatrexate or trimetrexate; spindle poisons including vinca alkaloids such as vinblastine, vincristine, vinorelbine and vindesine, or their synthetic analogues such as navelbine, or estramustine and a taxoid; platinum compounds such as cisplatin; and epipodophyllotoxins such as etoposide or teniposide.

As used herein, the tenn “apoptosis” refers to programmed cell death and is characterized by certain cellular characteristics such as membrane blebbing, chromatin condensation and fragmentation, or the formation of apoptotic bodies. Apoptosis is a genetically determined process of cell self-destruction that is marked by the fragmentation of nuclear DNA, is activated either by the presence of a stimulus or by the removal of a stimulus or suppressing agent, is a normal physiological process eliminating DNA-damaged, superfluous, or unwanted cells, and when halted (as, e.g., by genetic mutation), may result in uncontrolled cell growth and tumor formation.

The term “carbocycle,” as used herein, refers to an aromatic or nonaromatic ring in which each atom of the ring is carbon.

The term “carbonyl” is art-recognized and includes such moieties as can be represented by the general formula: 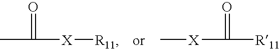
wherein X is a bond or represents an oxygen or a sulfur, and R₁₁ represents a hydrogen, an alkyl, an alkenyl, —(CH₂)ₘ—R₈ or a pharmaceutically acceptable salt, R′₁₁ represents a hydrogen, an alkyl, an alkenyl or —(CH₂)ₘ—R₈, where m and R₈ are as defined above. Where X is an oxygen and R₁₁ or R′₁₁ is not hydrogen, the formula represents an “ester.” Where X is an oxygen, and R₁₁ is as defined above, the moiety is referred to herein as a carboxyl group, and particularly when R₁₁ is a hydrogen, the formula represents a “carboxylic acid.” Where X is an oxygen, and R′₁₁ is hydrogen, the formula represents a “formate.” In general, where the oxygen atom of the above formula is replaced by sulfur, the formula represents a “thiocarbonyl” group. Where X is a sulfur and R₁₁ or R′₁₁ is not hydrogen, the formula represents a “thioester.” Where X is a sulfur and R₁₁ is hydrogen, the formula represents a “thiocarboxylic acid.” Where X is a sulfur and R₁₁′ is hydrogen, the formula represents a “thiolformate.” On the other hand, where X is a bond, and R₁₁ is not hydrogen, the above formula represents a “ketone” group. Where X is a bond, and R₁₁ is hydrogen, the above formula represents an “aldehyde” group.

The term “heteroatom” as used herein means an atom of any element other than carbon or hydrogen. Preferred heteroatoms are boron, nitrogen, oxygen, phosphorus, sulfur and selenium.

The terms “heterocyclyl” or “heterocyclic group” refer to 3- to 10-membered ring structures, more preferably 3- to 7-membered rings, whose ring structures include one to four heteroatoms. Heterocycles can also be polycycles. Heterocyclyl groups include, for example, thiophene, thianthrene, furan, pyran, isobenzofuran, chromene, xanthene, phenoxathiin, pyrrole, imidazole, pyrazole, isothiazole, isoxazole, pyridine, pyrazine, pyrimidine, pyridazine, indolizine, isoindole, indole, indazole, purine, quinolizine, isoquinoline, quinoline, phthalazine, naphthyridine, quinoxaline, quinazoline, cinnoline, pteridine, carbazole, carboline, phenanthridine, acridine, pyrimidine, phenanthroline, phenazine, phenarsazine, phenothiazine, furazan, phenoxazine, pyrrolidine, oxolane, thiolane, oxazole, piperidine, piperazine, morpholine, lactones, lactams such as azetidinones and pyrrolidinones, sultams, sultones, and the like. The heterocyclic ring can be substituted at one or more positions with such substituents as described above, as for example, halogen, alkyl, aralkyl, alkenyl, alkynyl, cycloalkyl, hydroxyl, amino, nitro, sulfhydryl, imino, amido, phosphate, phosphonate, phosphinate, carbonyl, carboxyl, silyl, ether, alkylthio, sulfonyl, ketone, aldehyde, ester, a heterocyclyl, an aromatic or heteroaromatic moiety, —CF₃, —CN, or the like.

The term “contacting” is used herein interchangeably with the following: combined with, added to, mixed with, passed over, incubated with, etc. Moreover, the compounds of the present invention can be “administered” by any conventional method such as, for example, parenteral, oral, topical and inhalation routes as described herein.

As used herein, the term “co-administration” or “co-administering” refers to administration of one component of the method, e.g., a hedgehog inhibitor, with another component, e.g., radiation and/or a chemotherapeutic agent, concurrently, i.e., simultaneously in time, or sequentially, i.e., administration of one component, followed by administration of the other component. That is, after administration of one component, the second component can be administered substantially immediately after the first component, or the second component can be administered after an effective time period after the first component, the effective time period being the amount of time given for realization of maximum benefit from the administration of the first component.

As used herein, “combination therapy” (or “co-therapy”) refers to the administration of the hedgehog inhibitor and radiotherapy, the hedgehog inhibitor and a chemotherapeutic agent, or the hedgehog inhibitor, radiotherapy and a chemotherapeutic agent during the course of cancer therapy. Such combination therapy may involve the administration of the hedgehog inhibitor before, during, and/or after the administration of the radiation therapy and/or chemotherapy. The administration of the hedgehog inhibitor may be separated in time from the administration of radiotherapy and/or chemotherapy by up to several weeks, and may precede it or follow it, but more commonly the administration of the hedgehog inhibitor will accompany at least one aspect of the radiation therapy and/or chemotherapy (such as the administration of one dose of radiation therapy and/or chemotherapy within up to 48 hours, and most commonly within less than 24 hours).

Combination therapy also can embrace the administration of the hedgehog inhibitor and radiation therapy and/or chemotherapy as described above in further combination with other biologically active agents or modalities such as, but not limited to, another antineoplastic agent and non-drug therapies (such as, but not limited to, surgery).

As used herein, “concurrently” means (1) simultaneously in time, or (2) at different times during the course of a common treatment schedule.

As used herein, the term “hedgehog inhibitor” refers to an agent capable of blocking cellular responses to the hedgehog signaling pathway, e.g., in cells with an active hedgehog signaling pathway, and more specifically, inhibiting cellular responses, directly or indirectly, to the hedgehog family of secreted growth factors. The hedgehog inhibitor may antagonize hedgehog pathway activity through a number of routes, including, but not limited to, by interfering with the inhibitory effect that Ptc exerts on Smo; by activating Smo without affecting Ptc; by influencing Smo function by directly binding to Smo; and/or by activating the pathway downstream of Smo. Exemplary hedgehog inhibitors may include, but are not limited to, steroidal alkaloids such as cyclopamine and jervine.

As used herein, the term “halogen” designates —F, —Cl, —Br or —I.

As used herein, the term “hydroxyl” means —OH.

As used herein, the term “nitro” means —NO₂

As used herein, “patient” refers to a mammal, preferably a human, in need of treatment for a condition, disorder or disease.

The term “phosphonamidite” can be represented in the general formula: 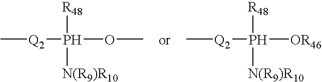
wherein R₉ and R₁₀ are as defined above, Q₂ represents O, S or N, and R₄₈ represents a lower alkyl or an aryl, Q₂ represents O, S or N.

A “phosphoramidite” can be represented in the general formula:

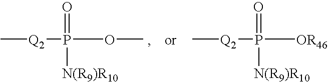

wherein R_{9 and R}₁₀ are as defined above, and Q₂ represents O, S or N.

A “phosphoryl” can in general be represented by the formula: 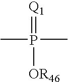
wherein Q₁ represented S or O, and R₄₆ represents hydrogen, a lower alkyl or an aryl. When used to substitute, for example, an alkyl, the phosphoryl group of the phosphorylalkyl can be represented by the general formula: 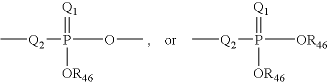
wherein Q₁ represented S or O, and each R₄₆ independently represents hydrogen, a lower alkyl or an aryl, Q₂ represents O, S or N. When Q₁ is an S, the phosphoryl moiety is a “phosphorothioate.”

The terms “polycyclyl” or “polycyclic group” refer to two or more rings (e.g., cycloalkyls, cycloalkenyls, cycloalkynyls, aryls and/or heterocyclyls) in which two or more carbons are common to two adjoining rings, e.g., the rings are “fused rings.” Rings that are joined through non-adjacent atoms are termed “bridged” rings. Each of the rings of the polycycle can be substituted with such substituents as described above, as for example, halogen, alkyl, aralkyl, alkenyl, alkynyl, cycloalkyl, hydroxyl, amino, nitro, sulfhydryl, imino, amido, phosphate, phosphonate, phosphinate, carbonyl, carboxyl, silyl, ether, alkylthio, sulfonyl, ketone, aldehyde, ester, a heterocyclyl, an aromatic or heteroaromatic moiety, —CF₃, —CN, or the like.

The phrase “protecting group” as used herein means temporary substituents which protect a potentially reactive fimctional group from undesired chemical transformations. Examples of such protecting groups include esters of carboxylic acids, silyl ethers of alcohols, and acetals and ketals of aldehydes and ketones, respectively. The field of protecting group chemistry has been reviewed (Greene, T. W.; Wuts, P. G. M., Protective Groups in Organic Synthesis, 2nd ed.; Wiley, N.Y. (1991)).

A “selenoalkyl” refers to an alkyl group having a substituted seleno group attached thereto. Exemplary “selenoethers” which may be substituted on the alkyl are selected from one of —Se-alkyl, —Se-alkenyl, —Se-alkynyl, and —Se—(CH₂)ₘ—R₈, m and R₈ being defined above.

As used herein, “sequentially” means administration of one component of the method, a hedgehog inhibitor, followed by administration of the other component, i.e., radiation; after administration of one component, the second component can be administered substantially immediately after the first component, or the second component can be administered after an effective time period after the first component; the effective time period is the amount of time given for realization of maximum benefit from the administration of the first component.

As used herein, the term “sulfhydryl” means —SH.

As used herein, the term “sulfonyl” means —SO₂—.

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. The permissible substituents can be one or more and the same or different for appropriate organic compounds. This invention is not intended to be limited in any manner by the permissible substituents of organic compounds.

The term “sulfamoyl” is art-recognized and includes a moiety that can be represented by the general formula: 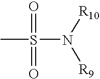
in which R₉ and R₁₀ are as defined above.

The term “sulfate” is art recognized and includes a moiety that can be represented by the general formula: 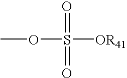
in which R₄₁, is as defined above.

The term “sulfonamido” is art recognized and includes a moiety that can be represented by the general formula:

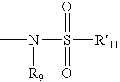

in which R_{9 and R′}₁₁ are as defined above.

The term “sulfonate” is art-recognized and includes a moiety that can be represented by the general formula:

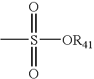

in which R₄₁ is an electron pair, hydrogen, alkyl, cycloalkyl, or aryl.

The terms “sulfoxido” or “sulfinyl,” as used herein, refers to a moiety that can be represented by the general formula: 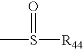
in which R₄₄ is selected from the group consisting of hydrogen, alkyl, alkenyl, alkynyl, cycloalkyl, heterocyclyl, aralkyl, or aryl.

Analogous substitutions can be made to alkenyl and alkynyl groups to produce, for example, aminoalkenyls, aminoalkynyls, amidoalkenyls, amidoalkynyls, iminoalkenyls, iminoalkynyls, thioalkenyls, thioalkynyls, carbonyl-substituted alkenyls or alkynyls.

As used herein, a “therapeutically effective amount” refers to that amount which, when administered to a mammal, especially a human, for treating a cancer, is sufficient to effect treatment for the cancer. Alternatively, a “therapeutically effective amount” is sufficient to cause an improvement in a clinically significant condition or symptom in a patient. “Effective amount” may also refer to that amount of an agent (i.e., chemical or radiative) that elicits the requisite biological or medical response in cells.

As used herein, “treating” or “treatment” of a cancer in a mammal includes one or more of: (1) inhibiting growth of the cancer, i.e., arresting its development, (2) preventing spread of the cancer, i.e., preventing metastases, (3) relieving the cancer, i.e., causing regression of the cancer, (4) preventing recurrence of the cancer, and (5) palliating symptoms of the cancer. “Treatment” refers to therapy, prevention and prophylaxis, and more particularly, refers to the administration of medicine or other modality or to the performance of medical procedures with respect to a patient, for either prophylaxis or to cure or reduce the extent of or likelihood of occurrence of the condition of which the patient is afflicted.

As used herein, the term “tumor” includes neoplasms that are identifiable through clinical screening or diagnostic procedures including, but not limited to, palpation, biopsy, cell proliferation index, endoscopy, mammography, digital mammography, ultrasonography, computed tomography (CT), magnetic resonance imaging (MRI), positron emission tomography (PET), radiography, radionuclide evaluation, CT- or MRI-guided aspiration cytology, and imaging-guided needle biopsy, among others. Such diagnostic techniques are well known to those skilled in the art and are described in Holland, et al., Cancer Medicine, 4th Ed., Vol. One, Williams & Wilkins, Baltimore, Md. (1997).

The term “ED₅₀” refers to the dose of a drug which produces 50% of its maximum response or effect.

Solid tumors that may be suitably treated with the methods of the present invention include, but are not limited to, tumors of the brain (glioblastomas, medulloblastoma, astrocytoma, oligodendroglioma, ependymomas), lung, liver, spleen, kidney, lymph node, small intestine, pancreas, blood cells, colon, stomach, breast, endometrium, prostate, testicle, ovary, skin, head and neck, esophagus, bone marrow, blood and other tissue. The tumor may be distinguished as metastatic and non-metastatic.

It also is specifically understood that any numerical value recited herein includes all values from the lower value to the upper value, i.e., all possible combinations of numerical values between the lowest value and the highest value enumerated are to be considered to be expressly stated in this application. For example, if a concentration range or a beneficial effect range is stated as 1% to 50%, it is intended that values such as 2% to 40%, 10% to 30%, or 1% to 3%, etc., are expressly enumerated in this specification. These are only examples of what is specifically intended.

Some embodiments of the invention provide a method of inhibiting growth of a cancer cell by contacting the cell with hedgehog inhibitor and a chemotherapeutic agent; the hedgehog inhibitor and the chemotherapeutic agent are each provided in an effective growth-inhibiting amount. The hedgehog inhibitor and chemotherapeutic agent may be administered to a human cancer patient in amounts which are effective to inhibit the growth of cancer. The methods of the present invention are particularly suitable to those malignant cells that express the hedgehog signaling pathway.

In an illustrated embodiment, the present invention provides a method of inhibiting the growth of pancreatic cancer cells. In other words, the method can form part of a treatment program for pancreatic cancer. Pancreatic cancer is a common malignancy with an extremely poor prognosis. Many pancreatic cancers express or overexpress the hedgehog signaling pathway.

The compounds of the present invention, particularly libraries of variants having various representative classes of substituents, are amenable to combinatorial chemistry and other parallel synthesis schemes (see, for example, PCT WO 94/08051). The result is that large libraries of related compounds, e.g. a variegated library of compounds represented above, can be screened rapidly in high throughput assays in order to identify potential hedgehog inhibitor compounds, as well as to refine the specificity, toxicity, and/or cytotoxic-kinetic profile of a potential inhibitor compound. For instance, ptc, hedgehog, or smoothened bioactivity assays, may be developed using cells with either a ptc loss-of-function, hedgehog gain-of-function, or smoothened gain-of-function, can be used to screen a library of the subject compounds for those having agonist activity toward ptc or antagonist activity towards hedgehog or smoothened. Alternatively, bioactivity assays using cells with either a ptc gain-of-function, hedgehog loss-of-function, or smoothened loss-of-function, can be used to screen a library of the subject compounds for those having antagonist activity toward ptc or agonist activity towards hedgehog or smoothened. See also, Williams et al., supra, for establishing screening systems for hedgehog inhibitors.

Simply for illustration, a combinatorial library for the purposes of the present invention is a mixture of chemically related compounds which may be screened together for a desired property. The preparation of many related compounds in a single reaction greatly reduces and simplifies the number of screening processes which need to be carried out. Screening for the appropriate physical properties can be done by conventional methods.

A variety of techniques are available in the art for generating combinatorial libraries of small organic molecules such as the subject hedgehog inhibitors. (See, for example, Blondelle et al., Trends Anal. Chem., 14:83 (1995); U.S. Pat. Nos. 5,359,115 and 5,362,899; U.S. Pat. No. 5,288,514; PCT publication WO 94/08051; U.S. Pat. Nos. 5,736,412 and 5,712,171; Chen et al., JACS, 116:2661 (1994); Kerr et al., JACS, 115:252 (1993); PCT publications WO 92/10092, WO 93/09668 and WO 91/07087; and PCT publication WO 93/20242, all of which are incorporated by reference in their entireties). Accordingly, a variety of libraries on the order of about 100 to 1,000,000 or more diversomers of the subject compounds can be synthesized and screened for particular activity or property.

Diversity in the library can be created at a variety of different levels. For instance, the substrate aryl groups used in the combinatorial reactions can be diverse in terms of the core aryl moiety, e.g., a variation in terms of the ring structure, and/or can be varied with respect to the other substituents.

In an exemplary embodiment, a library of candidate compound diversomers can be synthesized utilizing a scheme adapted to the techniques described in the Still et al. PCT publication WO 94/08051, incorporated herein by reference, e.g., being linked to a polymer bead by a hydrolyzable or photolyzable group, optionally located at one of the positions of the candidate regulators or a substituent of a synthetic intermediate. According to the Still et al. technique, the library is synthesized on a set of beads, each bead including a set of tags identifying the particular diversomer on that bead. The bead library can then be “plated” with, for example, ptc loss-of-function, hedgehog gain-of-function, or smoothened gain-of-function cells for which a hedgehog agonist is sought. The diversomers can be released from the bead, e.g., by hydrolysis.

Many variations on the above and related pathways permit the synthesis of widely diverse libraries of compounds which may be tested as regulators of hedgehog function.

Moreover, there are a variety of assays available for determining the ability of a compound such as a hedgehog regulator to regulate ptc, smoothened, or hedgehog function, many of which can be disposed in high-throughput formats. In many drug screening programs which test libraries of compounds and natural extracts, high throughput assays are desirable in order to maximize the number of compounds surveyed in a given period of time. Thus, libraries of synthetic and natural products can be sampled for other compounds which are hedgehog regulators.

In addition to cell-free assays, test compounds can also be tested in cell-based assays. In one embodiment, cell which have a ptc loss-of-function, hedgehog gain-of-function, or smoothened gain-of-function phenotype can be contacted with a test agent of interest, with the assay scoring for, e.g., inhibition of proliferation of the cell in the presence of the test agent.

A number of gene products have been implicated in patched-mediated signal transduction, including patched, transcription factors of the cubitus interruptus (ci) family, the serine/threonine kinase fused (fu) and the gene products of costal-2, smoothened and suppressor of fused.

The induction of cells by hedgehog proteins sets in motion a cascade involving the activation and inhibition of downstream effectors, the ultimate consequence of which is, in some instances, a detectable change in the transcription or translation of a gene. Potential transcriptional targets of hedgehog-mediated signaling are the patched gene (Hidalgo and Ingham, 1990 Development 110, 291-301; Marigo et al., 1996) and the vertebrate homologs of the drosophila cubitus interruptus gene, the GLI genes (Hui et al. (1994) Dev Biol 162:402-413). Patched gene expression has been shown to be induced in cells of the limb bud and the neural plate that are responsive to Shh. (Marigo et al. (1996) PNAS 93:9346-51; Marigo et al. (1996) Development 122:1225-1233). The Gli genes encode putative transcription factors having zinc finger DNA binding domains (Orenic et al. (1990) Genes & Dev 4:1053-1067; Kinzler et al. (1990) Mol Cell Biol 10:634-642). Transcription of the Gli gene has been reported to be upregulated in response to hedgehog in limb buds, while transcription of the Gli3 gene is downregulated in response to hedgehog induction Narigo et al. (1996) Development 122:1225-1233). By selecting transcriptional regulatory sequences from such target genes, e.g., from patched or Gli genes, that are responsible for the up- or down-regulation of these genes in response to hedgehog signaling, and operatively linking such promoters to a reporter gene, one can derive a transcription based assay which is sensitive to the ability of a specific test compound to modify hedgehog-mediated signaling pathways. Expression of the reporter gene, thus, provides a valuable screening tool for the development of compounds that act as regulators of hedgehog.

Reporter gene based assays of this invention measure the end stage of the above described cascade of events, e.g., transcriptional modulation. Accordingly, in practicing one embodiment of the assay, a reporter gene construct is inserted into the reagent cell in order to generate a detection signal dependent on ptc loss-of-function, hedgehog gain-of-function, smoothened gain-of-function, or stimulation by SHH itself. The amount of transcription from the reporter gene may be measured using any method known to those of skill in the art to be suitable. For example, mRNA expression from the reporter gene may be detected using RNAse protection or RNA-based PCR, or the protein product of the reporter gene may be identified by a characteristic stain or an intrinsic biological activity. The amount of expression from the reporter gene is then compared to the amount of expression in either the same cell in the absence of the test compound or it may be compared with the amount of transcription in a substantially identical cell that lacks the target receptor protein. Any statistically or otherwise significant decrease in the amount of transcription indicates that the test compound has in some manner agonized the normal ptc signal (or antagonized the gain-of-function hedgehog or smoothened signal), e.g., the test compound is a potential hedgehog antagonist.

In one aspect, hedgehog inhibitors in accordance with the present invention are suitably steroid alkaloids that inhibit Hh signaling, e.g., via direct interaction with the protein Smoothened. Particular hedgehog inhibitors are certain steroid alkaloids, e.g., cyclopamine and related compounds thereof. (See, e.g., U.S. Published Application 2004/00729914; and U.S. Published Application 2003/0013646.)

In certain embodiments, the steroidal alkaloid is represented in the general formula (I), or unsaturated forms thereof and/or seco-, nor- or homo-derivatives thereof: 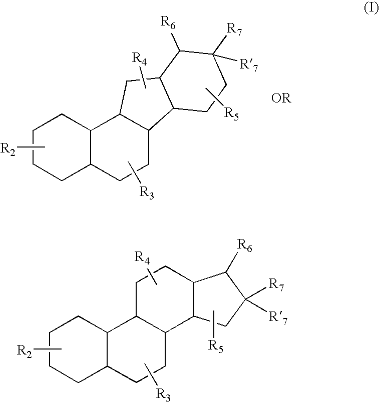
wherein, as valence and stability permit,
R₂, R₃, R₄, and R₅, represent one or more substitutions to the ring to which each is attached, for each occurrence, independently represent hydrogen, halogens, alkyls, alkenyls, alkynyls, aryls, hydroxyl, ═O, ═S, alkoxyl, silyloxy, amino, nitro, thiol, amines, imines, amides, phosphoryls, phosphonates, phosphines, carbonyls, carboxyls, carboxamides, anhydrides, silyls, ethers, thioethers, alkylsulfonyls, arylsulfonyls, selenoethers, ketones, aldehydes, esters, or —(CH₂)ₘ—R₈;
R₆, R₇, and R′₇, are absent or represent, independently, halogens, alkyls, alkenyls, alkynyls, aryls, hydroxyl, ═O, ═S, alkoxyl, silyloxy, amino, nitro, thiol, amines, imines, amides, phosphoryls, phosphonates, phosphines, carbonyls, carboxyls, carboxamides, anhydrides, silyls, ethers, thioethers, alkylsulfonyls, arylsulfonyls, selenoethers, ketones, aldehydes, esters, or —(CH₂)ₘ—R₈, or
R₆ and R₇, or R₇ and R′₇, taken together form a ring or polycyclic ring, e.g., which is substituted or unsubstituted, with the proviso that at least one of R₆, R₇, or R′₇ is present and includes a primary or secondary amine;
R₈ represents an aryl, a cycloalkyl, a cycloalkenyl, a heterocycle, or a polycycle; and m is an integer in the range 0 to 8 inclusive.

In particular embodiments, R₂ and R₃, for each occurrence, is an —OH, alkyl, —O-alkyl, —C(O)-alkyl, or —C(O)—R₈;
R₄, for each occurrence, is an absent, or represents —OH, ═O, alkyl, —O-alkyl, —C(O)-alkyl, or —C(O)—R₈;
R₆, R₇, and R′₇ each independently represent, hydrogen, alkyls, alkenyls, alkynyls, amines, imines, arnides, carbonyls, carboxyls, carboxamides, ethers, thioethers, esters, or —(CH₂)ₘ—R₈, or
R₇, and R′₇ taken together form a furanopiperidine, such as perhydrofuro[3,2-b]pyridine, a pyranopiperidine, a quinoline, an indole, a pyranopyrrole, a naphthyridine, a thiofuranopiperidine, or a thiopyranopiperidine with the proviso that at least one of R₆, R₇, or R′₇ is present and includes a primary or secondary amine;
R₈ represents an aryl, a cycloalkyl, a cycloalkenyl, a heterocycle, or a polycycle, and preferably R₈ is a piperidine, pyrimidine, morpholine, thiomorpholine, pyridazine,

In certain embodiments, the steroidal alkaloid is represented in the general formula (II), or unsaturated forms thereof and/or seco-, nor- or homo-derivatives thereof: 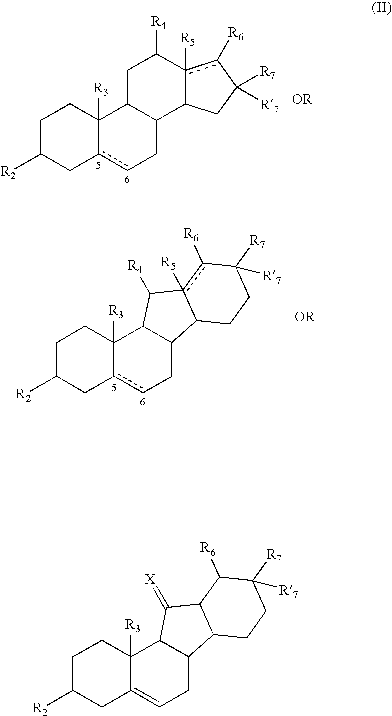
wherein R₂, R₃, R₄, R₅, R₆, R₇, and R′₇ are as defined above, and X represents O or S, though preferably O.

In certain embodiments, the steroidal alkaloid is represented in the general formula (III), or unsaturated forms thereof and/or seco-, nor- or homo-derivatives thereof: 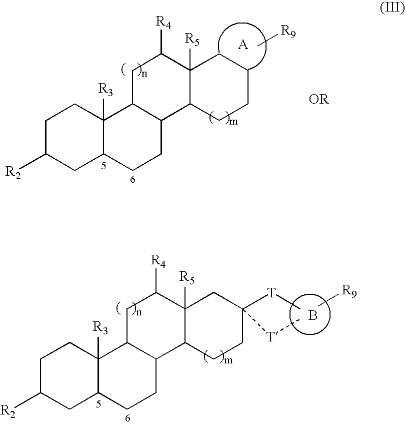
wherein
R₂, R₃, R₄, R₅ and R₈ are as defined above;
A and B represent mono cyclic or polycyclic groups;
T represent an alkyl, an aminoalkyl, a carboxyl, an ester, an amide, ether or amine linkage of 1-10 bond lengths;
T′ is absent, or represents an alkyl, an aminoalkyl, a carboxyl, an ester, an amide, ether or amine linkage of 1-3 bond lengths, wherein if T and T′ are present together, than T and T′ taken together with the ring A or B form a covelently closed ring of 5-8 ring atoms;
R₉ represent one or more substitutions to the ring A or B, which for each occurrence, independently represent halogens, alkyls, alkenyls, alkynyls, aryls, hydroxyl, ═O, ═S, alkoxyl, silyloxy, amino, nitro, thiol, amines, imines, amides, phosphoryls, phosphonates, phosphines, carbonyls, carboxyls, carboxamides, anhydrides, silyls, ethers, thioethers, alkylsulfonyls, arylsulfonyls, selenoethers, ketones, aldehydes, esters, or —(CH₂)ₘ—R₈; and
and m are, independently, zero, 1 or 2; with the proviso that A and R₉, or T, T′, B and R₉, taken together include at least one primary or secondary amine.

In certain embodiments, the steroidal alkaloid is represented in the general formula (IV), or unsaturated forms thereof and/or seco-, nor- or homo-derivatives thereof: 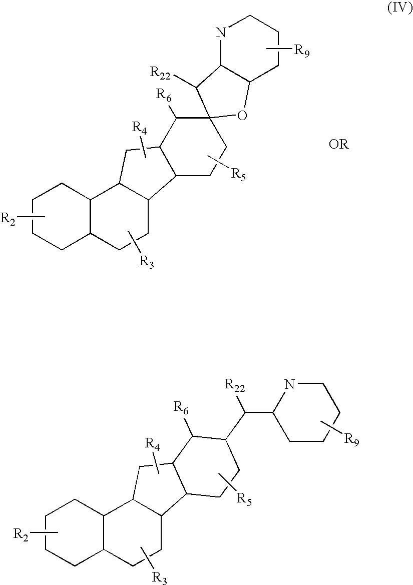
wherein
R₂, R₃, R₄, R₅, R₆ and R₉ are as defined above;
R₂₂ is absent or represents an alkyl, an alkoxyl or —OH.

In certain embodiments, the steroidal alkaloid is represented in the general formula (V) or unsaturated forms thereof and/or seco-, nor- or homo-derivatives thereof: 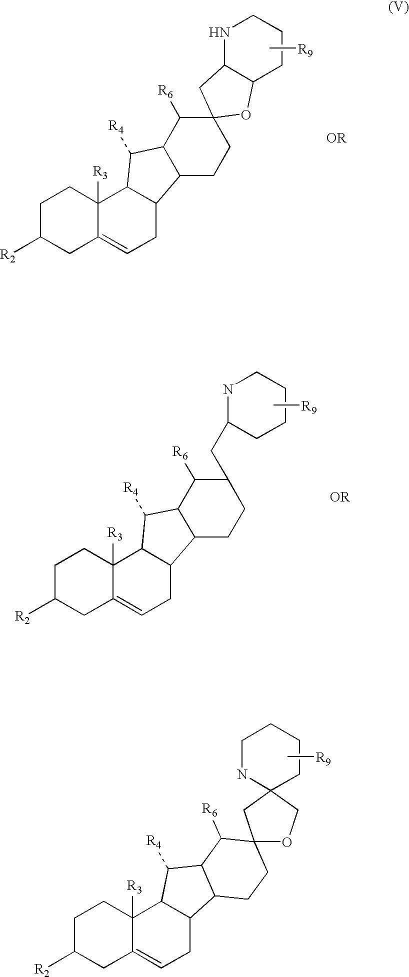
wherein
R₂, R₃, R₄, R₆ and R₉ are as define above;

In certain embodiments, the steroidal alkaloid is represented in the general formula (VI), or unsaturated forms thereof and/or seco-, nor- or homo-derivatives thereof: 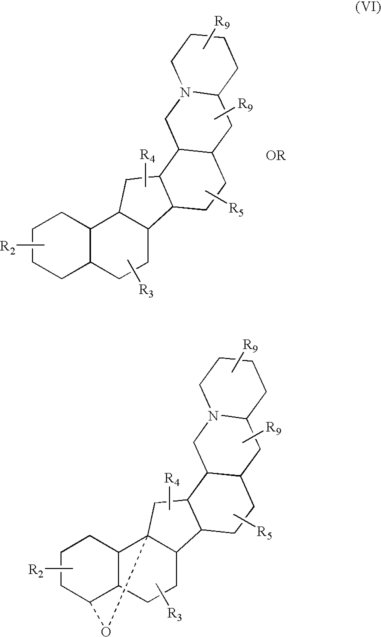
wherein R₂, R₃, R₄, R₅ and R₉ are as defined above;

In certain embodiments, the steroidal alkaloid is represented in the general formula (VII) or unsaturated forms thereof and/or seco-, nor- or homo-derivatives thereof: 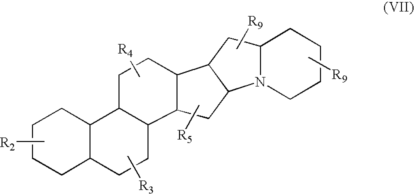
wherein R₂, R₃, R₄, R₅ and R₉ are as defined above.

Of particular interest are steroid alkaloids, which include derivatives of veratrum alkaloids, such as cyclopamine, veratramine, and jervine, shown below in Formula (VIII), wherein R¹, R², R³ and R⁴ are independently selected from hydrogen, alkyl, alkenyl, alkynyl, aryl, hydroxyl, alkoxy, carbonyl, carboxyl, ketones and aldehydes, and analogs and derivatives thereof. R represents one or more independent substitutions to the aryl group selected from hydrogen, alkyl, alkenyl, alkynyl, aryl, hydroxyl, alkoxy, carbonyl, carboxyl, ketones and aldehydes, and analogs and derivatives thereof.

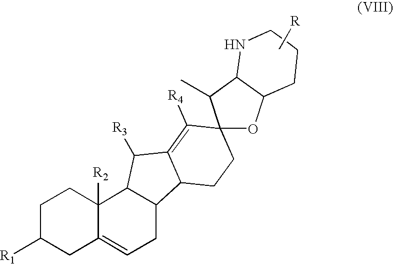

Of particular value may be cyclopamine. Cyclopamine (available from BIOMOL®, Plymouth Meeting, Pa.) is represented by Formula (IX).

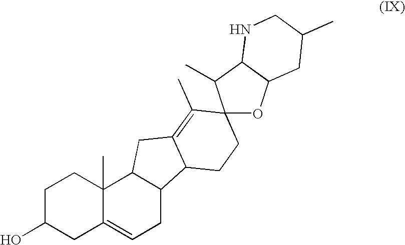

Also of particular value may be jervine. Jervine (available from BIOMOL®, Plymouth Meeting, Pa.) is represented by Formula (X).

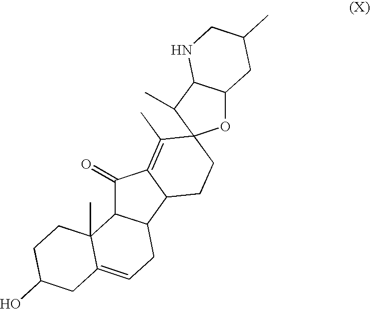

Other suitable hedgehog inhibitors include small molecule inhibitors as described in, e.g., Williams, et al., Proc. Nat'l Acad. Sci. USA, 100, 4616-4621 (2003); Gabay et al., Neuron, 40, 485-499 (e.g., certain benzimidazole compounds); U.S. Pat. No. 6,613,798; U.S. Pat. No. 6,545,005; U.S. Pat. No. 6,432,970; U.S. Pat. No. 6,291,516; Romer et al., Cancer Cell, 6, 229-240; U.S. Pat. No. 6,552,016; U.S. Pat. No. 6,683,108; and U.S. Pat. No. 6,686,388, all of which are incorporated by reference in their entireties.

It has been recently reported that prostate cancer xenografts NEJM MGH undergo complete regression after high dose cylopamine treatment. Cyclopamine has also demonstrated anti-tumor effects in murine tumor allografts of medulloblastoma. In pancreatic cancer cell lines with over-activation of Hh pathway signaling, cyclopamine induced apoptosis, while other pancreas cell lines were resistant. Although several studies have shown a cytotoxic effect of cyclopamine on various tumor cells that over express hedgehog pathway proteins, the potential use of cyclopamine as a single agent for treatment of cancer, e.g., pancreatic cancer, is limited by the heterogeneity of tumor population, differential tumor cell sensitivity, and high production costs.

It has been found that the instant hedgehog inhibitor compounds are particularly useful when co-administered with chemotherapy and/or radiation therapy. In other words, therapeutic combinations are contemplated wherein the hedgehog inhibitor is co-administered with a chemotherapeutic agent, such as taxol, and/or with radiation therapy.

In some embodiments, the chemotherapeutic agents are antimicrotubule agents. Paclitaxel (TAXOL®, available from Integrated BioPharma Inc., herein referred to as “Taxol”) is an antimicrotubule agent that promotes the assembly and stabilization of microtubules. This stability inhibits the normal reorganization of the microtubule network that is essential for vital interphase and mitotic cellular functions. It is contemplated that association of hedgehog pathway Gli proteins with microtubules during nuclear cyctoplasmic localization may permit taxol to enhance antitumor effects of inhibitors of the hedgehog pathway, e.g., cyclopamine.

Other classes of chemotherapeutic agents may also be of value, e.g., alkylating agents and antimetabolite agents. Cisplatin (PLATINOL®, Bristol-Myers Squibb Co., New York, N.Y.) is an alkylating agent that forms covalent bonds with guanine present in DNA. This action results in the formation of inter- and intra- chain cross linking which interferes with cellular transcription machinery and proliferation. Regulatory mechanisms detect the abnormal DNA and activate a cascade of responses to correct it, ultimately resulting in cell death via apoptosis.

Gemcitabine (GEMZAR®, Eli Lilly & Co.) is an antimetabolite agent which targets cells undergoing DNA synthesis (S phase) and blocks G1-S phase progression. Gemcitabine is actively metabolized by cellular nucleoside kinases to diphosphate (dFdCDP) and triphosphate (dFdCTP) nucleosides. Gemcitabine inhibits DNA synthesis by two mechanisms. First, gemcitabine diphosphate inhibits ribonucleotide reductase which is responsible for the generation of deoxynucleoside triphosphate for DNA sythesis, and second, gemcitabine competes with dCTP for incorporation into DNA. Gemcitabine is one of the recommended chemotherapeutic agents in advanced and metastatic pancreatic cancer.

Irradiation (or radiotherapy or radiation therapy) is used alone or in combination with chemotherapeutic agents and surgery for treatment of a variety of malignancies. Irradiation affects DNA either directly or via radiolysis of water and generation of reactive oxygen species. Irradiation causes DNA strand breaks, modified bases, abasic sites, sugar alterations, and DNA-protein cross-links. It is envisioned that combining the DNA damaging effects of irradiation and the inhibition of the hedgehog pathway by cyclopamine may enhance the antitumor effect of each of these single agents.

It is anticipated that hedgehog inhibitors used in combination with anticancer agents, i.e., chemotherapeutic drugs and/or radiation therapy, can give rise to a significantly enhanced cytotoxic effect on cancerous cells, thus providing an increased therapeutic effect. Specifically, as a significantly increased growth-inhibitory effect is obtained with the above disclosed combinations utilizing lower concentrations of the anticancer agents compared to the treatment regimes in which the agents are used alone, there is the potential to provide therapy wherein adverse side effects associated with the anticancer agents are considerably reduced than normally observed when anticancer agents are used alone in larger doses. By reducing the incidence of adverse effects, an improvement in the quality of life of a patient undergoing treatment for cancer is contemplated. Further, lowering the incidence of adverse effects may improve patient compliance and reduce the number of hospitalizations needed for the treatment of adverse effects.

The therapeutics of the invention can be tested in vivo for the desired therapeutic or prophylactic activity, as well as for determination of therapeutically effective dosage. For example, such compounds can be tested in suitable animal model systems prior to testing in humans, including, but not limited to, rats, mice, chicken, cows, monkeys, rabbits, etc. For in vivo testing, prior to administration to humans, any animal model system known in the art may be used.

Cyclopamine, as an exemplary hedgehog inhibitor, may be prepared as formulations at a pharmacologically effective dose in pharmaceutically acceptable media, for example, normal saline, PBS, etc. The additives may include bacteriocidal agents, stabilizers, buffers, or the like. Formulation of drugs is discussed in, for example, Hoover, John E., Remington's Pharmaceutical Sciences, Mack Publishing Co., Easton, Pa. (1975); and Liberman, H. A. and Lachman, L., Eds., Pharmaceutical Dosage Forms, Marcel Decker, New York, N.Y. (1980).

Actual dosage levels of the active ingredients in the pharmaceutical compositions of this invention may be varied so as to obtain an amount of the active ingredient which is effective to achieve the desired therapeutic response for a particular patient, composition, and mode of administration, without being toxic to the patient. The selected dosage level will depend upon a variety of factors including the activity of the particular compound of the present invention employed, or the ester, salt or arnide thereof, the route of administration, the time of administration, the rate of excretion of the particular compound being employed, the duration of the treatment, other drugs, compounds and/or materials used in combination with the particular hedgehog inhibitor employed, the age, sex, weight, condition, general health and prior medical history of the patient being treated, and like factors well known in the medical arts.

A physician having ordinary skill in the art can readily determine and prescribe the effective amount of the pharmaceutical composition required. For example, the physician could start doses of the hedgehog inhibitor compounds of the invention employed in the pharmaceutical composition at levels lower than that required in order to achieve the desired therapeutic effect and gradually increase the dosage until the desired effect is achieved.

Hedgehog inhibitors may be administered in a variety of routes, including orally, parenterally, intraperitoneally, intravenously, intraarterially, transdermally, sublingually, intramuscularly, rectally, transbuccally, intranasally, liposomally, via inhalation, vaginally, intraoccularly, via local delivery by catheter or stent, subcutaneously, intraadiposally, intraarticularly, intrathecally, or in a slow release dosage form. The hedgehog inhibitor is suitably administered orally.

Hedgehog inhibitors may be administered in an amount effective to cause arrest or regression of the cancer in a host when radiation and/or chemotherapy are also administered to the host. More suitably, a hedgehog inhibitor may be administered in an amount effective to achieve a serum level of at least about 2.0 micrograms/milliliter, still more suitably at least about 3.0 micrograms/milliliter. When administering a hedgehog inhibitor orally, a dosage is suitably at least about 5 mg/kg/day, more suitably at least about 10 mg/kg/day. Oral doses of hedghog inhibitor may be administered once or more than once per day. If oral doses are administered more than once per day, a suitable number of doses is three doses per day. If administering a hedgehog inhibitor intravenously, a preferable dosage is 10 mg/kg continuously. Intravenous dosage is suitably 3.3 mg/kg three times per day for a non-continuous (i.e., limited) period, such as two hours. Hedgehog inhibitors may be administered intravenously using a conventional non-saline infusion fluid, such as 5% dextrose in water. Hedgehog inhibitor dosing schedules may be for a variety of time periods, for example up to six weeks, or as determined by one of ordinary skill in the art to which this invention pertains.

The amount of radiation and/or chemotherapy delivered to the desired treatment volume may be variable. Radiation and/or chemotherapy may be administered in a dose effective to cause the arrest or regression of the cancer in a host, when the radiation and/or chemotherapy is administered with a hedgehog inhibitor.

Radiation may be administered in a variety of fashions. For example, radiation may be electromagnetic or particulate in nature. Electromagnetic radiation useful in the practice of this invention includes, but is not limited to, x-rays and gamma rays. Particulate radiation useful in the practice of this invention includes, but is not limited to, electron beams, proton beams, neutron beams, alpha particles, and negative pi mesons. The radiation may be delivered using conventional radiological treatment apparatus and methods, and by intraoperative and stereotactic methods. Additional discussion regarding radiation treatments suitable for use in the practice of this invention may be found throughout Steven A. Leibel et al., Textbook of Radiation Oncology, W. B. Saunders Co. (1998), and particularly in Chapters 13 and 14. Radiation may also be delivered by other methods such as targeted delivery, for example by radioactive “seeds,” or by systemic delivery of targeted radioactive conjugates. Other radiation delivery methods may also be used in the practice of this invention.

The amount of radiation delivered to the desired treatment volume may be variable. Radiation may suitably be administered in amount effective to cause the arrest or regression of the cancer in a host, when the radiation is administered with a hedgehog inhibitor and/or a chemotherapeutic agent. For example, radiation is suitably administered in at least about 1 Gray (Gy) fraction at least once every other day to a treatment volume, more suitably radiation is administered in at least about 2 Gy fractions at least once per day to a treatment volume, and even more suitably radiation is administered in at least about 2 Gy fractions at least once per day to a treatment volume for five consecutive days per week. In another embodiment, radiation is suitably administered in 3 Gy fractions every other day, three times per week to a treatment volume. In yet another embodiment, a total of at least about 20 Gy, or suitably at least about 30 Gy, or more suitably at least about 60 Gy of radiation, is administered to a host in need thereof.

The amount of the chemotherapeutic agent delivered to the patient may be variable. In a suitable embodiment, the chemotherapeutic agent may be administered in an amount effective to cause arrest or regression of the cancer in a host, when the chemotherapy is administered with a hedgehog inhibitor and/or radiation therapy. For example, taxol may be administered intravenously in an amount of about 175 mg/m² over a continuous period, such as 3 hours, every 3 weeks. In another embodiment, taxol is suitably administered intravenously in an amount of about 135 mg/m² over a continuous period of 3 hours every three weeks. Another intravenous dosage is suitably about 100 mg/m² over 3 hours every 2 weeks. Chemotherapy dosing schedules may be for a variety of time periods, for example, up to once every 3 weeks for a total of four courses of treatment, or as determined by one of ordinary skill in the art to which this invention pertains.

The following examples are included to demonstrate preferred embodiments of the invention. It should be appreciated by those of skill in the art that the techniques disclosed in the examples which follow represent techniques discovered by the inventors to function well in the practice of the invention, and thus, can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### General Materials and Methods

### EXAMPLE 1

### Cell Culture and Cell Lines

MIA PaCa-2, BxPC-3, and HCT 116 cells were obtained from American Type Culture Collection (MIA PaCa-2, CLR-1420™; BxPC-3, CLR-1687™; HCT 116, CCL-247™; human cell lines, ATCC®, Rockville, Md.). Mia PaCa-2 cells were grown in DMEM high glucose, and supplemented with L-glutamine, 10% fetal bovine serum (FBS), and penicillin/streptomycin 1%. BxPC-3 cells were maintained in RPMI 1640 medium supplemented with 10% FBS and antibiotics. HCT 116 cell were maintained in MEM medium supplemented with 10% fetal bovine serum (FBS) and L-glutamine.

### EXAMPLE 2

### Colony Formation Assay

250 to 1000 cells were plated in 60 mm dishes. Cells were incubated overnight. At twenty four hours cells were irradiated (3.5 Gy). Cyclopamine 2-10 μMol (Toronto Research Chemicals, TRC, supplier, Canada) was added to culture media, or combination of both. For chemotherapeutic agents, the drug was added to culture media at appropriate concentrations 24 hours after plating. Cultures were incubated for 10-14 days. After incubation, cells were fixed and stained with 0.25% crystal violet, and colonies containing more than 50 cells were counted. Plating efficiency was normalized compared to control.

### EXAMPLE 3

### PLDR Measurement

Cells were grown to confluence in 60 mm dishes and maintained confluent for three days. Cyclopamine 10 μMol was added to media 12 hours before irradiation. After exposure to irradiation (3.5 Gy), cultures were trypsinized and plated for survival assay in multiple time points within 24 hours.

### EXAMPLE 4

### Annexin V-PE Assay

Cyclopamine 4 μmol was added to culture media of exponentially growing cells with or without chemotherapeutic agents and irradiated at 12 hours. Cells were trypsinized after 24, 48 or 72 hours. Annexin levels were measured (Annexin V: PE Apoptosis Detection Kit, BD Biosciences Pharningen™, San Jose, Calif.) for 0.5×10⁶ freshly detached cells. The presence of membrane permeabilization was monitored by 7-AAD (7-Amino-actinomycinD) staining per manufacturer's protocol. Cells were subsequently analyzed by FACScan (BD FACSCanto™, BD Biosciences Immunocytometry Systems™, San Jose, Calif. with the use of CellQuest software (BD CellQuest™ Pro, BD Biosciences Immunocytometry Systems™, San Jose, Calif. The percentage of apoptotic cells was calculated by scoring for cells positive for either annexin alone (early apoptotic) or both annexin and 7-AAD (late apoptotic). All experiments were done in triplicate.

### Studies

### EXAMPLE 5

### Effect of Cyclopamine, 3.5 Gy of Radiation, or a Combination of Both, on Tumor Cells

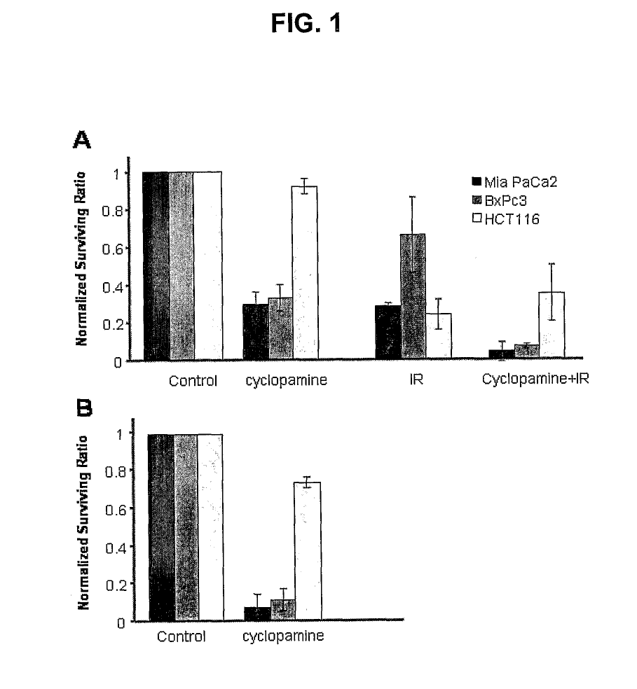

Testing was done as to whether cyclopamine is cytotoxic to hedgehog expressing pancreatic tumor cells, Mia PaCa-2 and BxPC-3, compared with human colon cancer cells, HCT 116, which do not express hedgehog. FIG. 1(A) contains a graph demonstrating the normalized surviving ratio in two pancreatic cell lines (Mia PaCa-2 and BxPC-3) and one colon cancer cell line (HCT 116) following exposure to 4 μMol of cyclopamine, 3.5 Gy of radiation, or a combination of both. Mia PaCa-2, BxPC-3, and HCT 116 had a 29%, 33% and 92% survival respectively. Next the effects of cyclopamine and IR were studied. 3.5 Gy radiation yielded 28%, 66%, and 24% survival in Mia PaCa-2, BxPC-3, and HCT 116 respectively. Cyclopamine 4 μM and irradiation demonstrated 4% survival in Mia PaCa-2, 7% survival in BxPC-3 and 35% survival in HCT 116 cells. Survival was measured by colony formation. P values for cyclopamine plus irradiation vs. irradiation alone is <0.05 for Mia PaCa-2 and BxPC-3 cell lines. These data demonstrate that cyclopamine is preferentially cytotoxic to Hh expressing pancreatic tumor cells compared with non-Hh expressing cells.

### EXAMPLE 6

### Effect of Cyclopamine on Colony Formation of Pancreatic and Colon Cancer Cell Lines

Testing was done as to whether cyclopamine is cytotoxic to hedgehog expressing pancreatic tumor cells, Mia PaCa-2 and BxPC-3, compared with human colon cancer cells, HCT 116, which do not express hedgehog. FIG. 1(B) demonstrates pancreatic and colon cancer cell lines colony formation following the exposure to 10 μMol cyclopamine in culture media. At 10 μM cyclopamine Mia PaCa-2 and BxPC-3 demonstrated 7% and 11% survival whereas HCT 116 demonstrated 74% survival. P<0.001. These data demonstrate that cyclopamine is preferentially cytotoxic to Hh expressing pancreatic tumor cells compared with non-Hh expressing cells.

### EXAMPLE 7

### Effect of Taxol, Cisplatin and Gemcitabine on Colony Formation

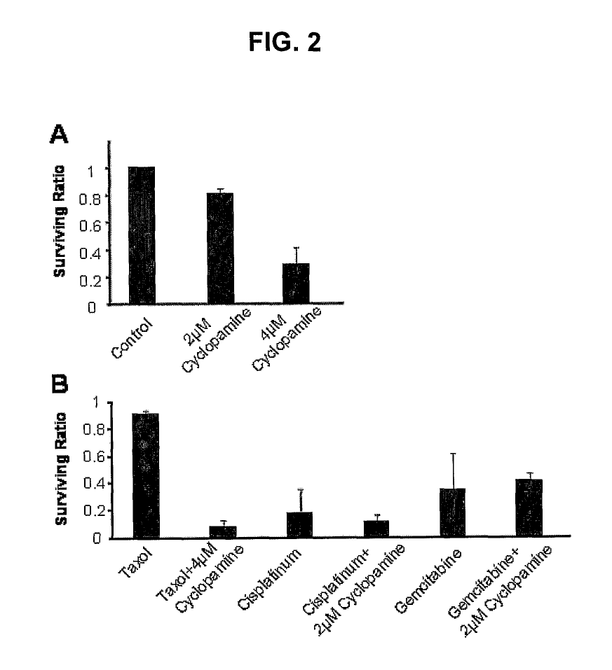

Testing was done to investigate the potential cytotoxic interaction between cyclopamine and chemotherapeutic agents. FIG. 2 shows colony formation following exposure to cyclopamine, taxol (3.5 nM), cisplatin (0.8 μMol), and gemcitabine (7.3 nM). In Mia PaCa-2 cells, cyclopamine (4 μM) gave a survival of 29% and taxol (3.5 nM) demonstrated survival of 91%. The combination of taxol (3.5 nM) and cyclopamine (4 μM) yielded a survival of 7% (p<0.001). Cisplatin alone (0.8 μM) gave 35% survival, and the combination of cyclopamine (2 μM) and cisplatin (0.8 μM) gave 11% survival. P=0.56. Gemcitabine (7.3 nM) demonstrated 35% survival and in combination with cyclopamine (2 μM) demonstrated 41% survival. P=0.7. Considered together, these data suggest a greater than additive effect between taxol and cyclopamine, an additive effect with cisplatin and cyclopamine, and a potentially protective effect between cyclopamine and gemcitabine.

### EXAMPLE 8

### Effect of Cyclopamine, Taxol, or a Combination of Both, on Apoptosis

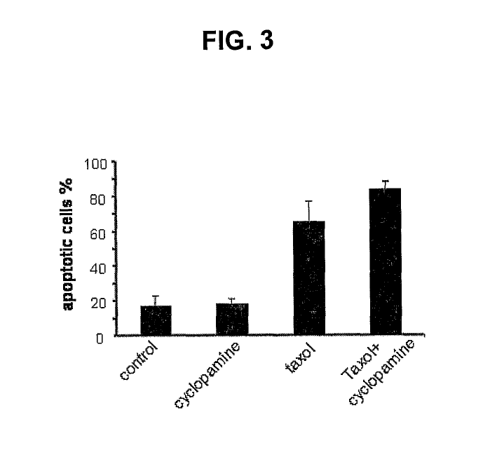

To test whether an increase in apoptosis accounted for the interactive killing between taxol and cyclopamine, Mia PcCa-2 cells were exposed to taxol (1.7 nM), or a combination of taxol (1.7 nM) and cyclopamine (4 μM), for 24 hours. FIG. 3 illustrates the percentage of apoptotic cells following exposure to cyclopamine (4 μMol), taxol (1.7 nMol), or a combination of both for 24 hours (AnnexinV-PE Assay). Apoptosis was measured by AnnexinV staining. Taxol induced 64.9% apoptosis, whereas the combination of taxol and cyclopamine demonstrated 83.5% apoptosis (compared to 18.2% in the cyclopamine group). These data suggest that some of the interactive killing between taxol and cyclopamine is due in part to an increase in apoptosis.

### EXAMPLE 9

### Effect of Cyclopamine, Gy Radiation, or a Combination of Both, on Apoptosis

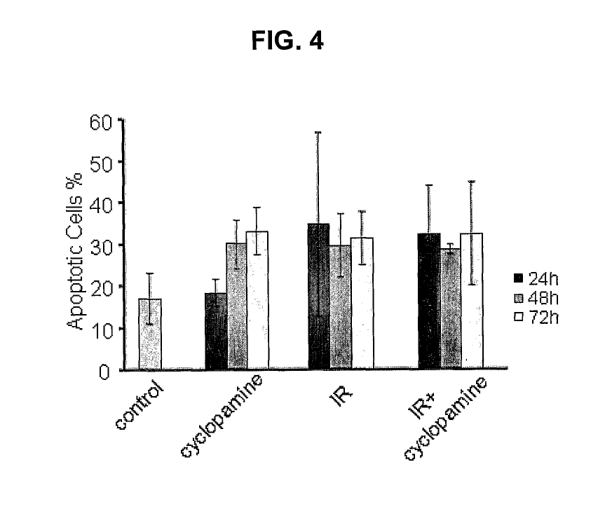

The combination of cyclopamine and radiation enhanced radiation killing by increasing apoptosis was measured by AnnexinV assay. FIG. 4 illustrates the percentage of apoptotic cells following exposure to cyclopamine (4 μMol), radiation (3.5 Gy), or a combination of both in 24, 48, or 72 hours (Annexin Assay). Cyclopamine induced apoptosis in 18.15%, 29.8%, and 32.9% of cells at 24, 48, and 72 hours, respectively. The baseline apoptotic rate in the control group was 16.92%. Apoptosis following 3.5 Gy radiation was 34.6%, 29.5% and 31.2% at 24, 48, and 72 hours, respectively. Apoptosis following exposure to a combination of radiation and cyclopamine was not significantly different from radiation alone (32.11%, 28.5%, and 32.3% at 24, 48, and 72 hours, respectively). These data considered together demonstrate that cyclopamine has an additive cytotoxic effect when combined with irradiation in hedgehog expressing tumor cells not accounted for by an increase in apoptosis. In cells that do not express the hedgehog pathway, cyclopamine did not have a significant effect on survival following irradiation.

### EXAMPLE 10

### Effect of Jervine, Gy Radiation, or a Combination of Both, on Apoptosis

The combination of jervine and radiation enhanced radiation killing by increasing apoptosis is measured by Annexin V assay in which the percentage of apoptotic cells following exposure to jervine (4 μMol), radiation (3.5 Gy), or a combination of both, in 24, 48, or 72 hours is determined. A baseline apoptotic rate is determined in a control group. The results demonstrate that jervine induces apoptosis in an increasing percentage of cells in a time-dependent manner. Apoptosis following 3.5 Gy radiation is determined as a function of time. Apoptosis following exposure to a combination of radiation and jervine is determined and found to differ little from radiation alone. These data considered together demonstrate that jervine has an additive cytotoxic effect when combined with irradiation in hedgehog expressing tumor cells not accounted for by an increase in apoptosis.

### EXAMPLE 11

### Effect of Cyclopamine, Radiation and Taxol on Apoptosis

A study is conducted to determine the increase in apoptosis with a combination of cyclopamine, taxol and radiation using the Annexin V assay as detailed in the above examples. The results show an increase in the percentage of apoptotic cells in a time dependent manner.

### EXAMPLE 12

### Effect of Jervine, Taxol, or a Combination of Both, on Apoptosis

To test whether an increase in apoptosis accounted for the interactive killing between taxol and jervine, Mia PcCa-2 cells are exposed to taxol (1.7 nM), or a combination of taxol (1.7 nM) and jervine (4 μM), for 24 hours. Apoptosis is measured by AnnexinV staining. The results demonstrate that the combination of taxol and jervine have a greater apoptotic effect on the cells than taxol alone, and significantly greater than jervine alone. These data suggest that some of the interactive killing between taxol and jervine is due in part to an increase in apoptosis.

### EXAMPLE 13

### Effect of Jervine, Radiation and Taxol on Apoptosis

A study is conducted to determine the increase in apoptosis with a combination of jervine, taxol and radiation using the Annexin V assay as detailed in the above examples. The results show an increase in the percentage of apoptotic cells in a time dependent manner.

While the present invention has now been described and exemplified with some specificity, those skilled in the art will appreciate the various modifications, including variations, additions, and omissions, which may be made in what has been described.

What is claimed is:

1. A method of inhibiting growth of cancer cells expressing the hedgehog signaling pathway comprising, contacting the cells with effective amounts of a hedgehog inhibitor and a chemotherapeutic agent to inhibit the growth of the cells.

2. The method of claim 1, wherein the cells are contacted with the hedgehog inhibitor and the chemotherapeutic agent concurrently or sequentially.

3. The method of claim 1, wherein the hedgehog inhibitor is a steroid alkaloid.

4. The method of claim 3, wherein the hedgehog inhibitor is a steroid alkaloid of formula (I): 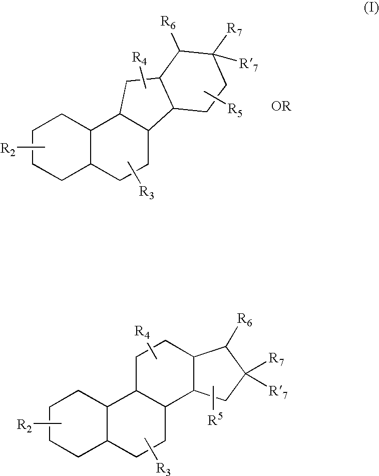
wherein, as valence and stability permit,
R₂, R₃,R₄, and R₅, represent one or more substitutions to the ring to which each is attached, for each occurrence, independently represent hydrogen, halogens, alkyls, alkenyls, alkynyls, aryls, hydroxyl, ═O, ═S, alkoxyl, silyloxy, amino, nitro, thiol, amines, imines, amides, phosphoryls, phosphonates, phosphines, carbonyls, carboxyls, carboxamides, anhydrides, silyls, ethers, thioethers, alkylsulfonyls, arylsulfonyls, selenoethers, ketones, aldehydes, esters, or —(CH₂)ₘ—R₈;
R₆, R₇, and R′₇, are absent or represent, independently, halogens, alkyls, alkenyls, alkynyls, aryls, hydroxyl, ═O, ═S, alkoxyl, silyloxy, amino, nitro, thiol, amines, imines, amides, phosphoryls, phosphonates, phosphines, carbonyls, carboxyls, carboxamides, anhydrides, silyls, ethers, thioethers, alkylsulfonyls, arylsulfonyls, selenoethers, ketones, aldehydes, esters, or —(CH₂)ₘ—R₉, or
R₆ and R₇, or R₇ and R′₇, taken together form a ring or polycyclic ring, e.g., which is substituted or unsubstituted, with the proviso that at least one of R₆, R₇, or R′₇ is present and includes a primary or secondary amine;
R₈ represents an aryl, a cycloalkyl, a cycloalkenyl, a heterocycle, or a polycycle;
and m is an integer in the range 0 to 8 inclusive.

5. The method of claim 3, wherein the steroid alkaloid is cyclopamine.

6. The method of claim 3, wherein the steroid alkaloid is jervine.

7. The method of claim 1, wherein the chemotherapeutic agent is an antimicrotubule agent, an alkylating agent, or an antimetabolite.

8. The method of claim 1, wherein the chemotherapeutic agent is selected from the group consisting of taxol, gemcitabine, cisplatin, and combinations thereof.

9. The method of claim 8, wherein the chemotherapeutic agent is taxol.

10. The method of claim 1, wherein effective amounts of the hedgehog inhibitor and the chemotherapeutic agent are co-administered to a mammalian cancer patient.

11. The method of claim 10, wherein the co-administration results in an increased sensitivity of the cells to cell apoptosis.

12. The method of claim 1, further comprising co-administering an effective dose of radiation.

13. The method of claim 10, wherein the hedgehog inhibitor is administered to the patient orally, intravascularly, subcutaneously, or peritoneally.

14. The method of claim 13, wherein the hedgehog inhibitor is administered to the patient daily, semi-weekly, biweekly, or weekly.

15. The method of claim 5, wherein the cyclopamine is administered to a mammalian cancer patient in a dosage of at least about 5 mg per kilogram of body weight per day.

16. A method of enhancing the antiproliferative effect of chemotherapy in a mammalian patient comprising, co-administering to the patient therapeutically effective amounts of a hedgehog inhibitor and a chemotherapeutic agent to enhance the antiproliferative effect of the chemotherapy.

17. The method of claim 16, wherein the hedgehog inhibitor is cyclopamine.

18. A method of inhibiting abnormal growth of cells expressing the hedgehog signaling pathway in a mammalian patient comprising, co-administering therapeutically effective amounts of a hedgehog inhibitor and a chemotherapeutic agent to inhibit the abnormal growth of the cells.

19. A method of inhibiting or reducing the growth of a tumor in a mammalian patient comprising, co-administering therapeutically effective amounts of a hedgehog inhibitor and a chemotherapeutic agent wherein the co-administration inhibits or reduces the ability of the tumor to grow.

20. The method of claim 19, wherein the tumor is a solid tumor or a blood-borne tumor.

21. The method of claim 19, wherein the mammalian patient has prostate cancer, lung cancer, breast cancer, colorectal cancer, or pancreatic cancer.

22. A therapeutic combination for inhibiting or reducing the proliferation of cancerous cells expressing the hedgehog signaling pathway comprising effective amounts of a hedgehog inhibitor and a chemotherapeutic agent to inhibit or reduce the proliferation of the cancerous cells.

23. A method of inhibiting the proliferation of cancerous cells expressing the hedgehog signaling pathway comprising, contacting the cells with therapeutically effective amounts of a hedgehog inhibitor and a chemotherapeutic agent, and an effective dose of radiation to inhibit or reduce the proliferation of the cells.

24. The method of claim 23, wherein the hedgehog inhibitor, the chemotherapeutic agent, and the radiation are co-administered to a mammalian cancer patient.

25. The method of claim 24, wherein the hedgehog inhibitor is a steroid alkaloid.

26. The method of claim 25, wherein the steroid alkaloid is cyclopamine.

27. The method of claim 26, wherein the cyclopamine is administered to the patient in a dosage of at least about 5 mg per kilogram of body weight per day.

28. The method of claim 26, wherein the cyclopamine is administered to the patient in an amount effective to achieve a serum level of at least about 2 μg/mL in the patient.

29. The method of claim 23, wherein the chemotherapeutic agent is taxol.

30. The method of claim 29, wherein the taxol is administered to a mammalian cancer patient in a dosage of about 100 to about 175 mg/m².

31. The method of claim 30, wherein the taxol is administered to the patient in a dosage of about 135 mg/m² over about 3 hours every two weeks.

32. The method of claim 24, wherein the radiation is administered to the patient in a dosage of at least about 1 Gray fraction once per day.

33. The method of claim 32, wherein the radiation is gamma radiation.

34. The method of claim 32, wherein the radiation is x-radiation.

35. A therapeutic combination for inhibiting or reducing the proliferation of cancerous cells expressing the hedgehog signaling pathway comprising therapeutically effective amounts of a hedgehog inhibitor and a chemotherapeutic agent, and an effective dose of radiation to inhibit or reduce the proliferation of the cells.

36. A method of inhibiting or reducing the proliferation of cancerous cells expressing the hedgehog signaling pathway comprising, contacting the cells with or introducing into the cells an effective amount of a hedgehog inhibitor and an effective dose of radiation to inhibit or reduce the proliferation of the cells.

37. The method of claim 36, wherein the cancerous cells comprise cells of prostate cancer, lung cancer, breast cancer, colorectal cancer, or pancreatic cancer.

38. The method of claim 36, wherein the hedgehog inhibitor and the radiation are co-administered to a mammalian cancer patient.

39. The method of claim 38, wherein the hedgehog inhibitor is a steroid alkaloid.

40. The method of claim 39, wherein the steroid alkaloid is cyclopamine.

41. A therapeutic combination for inhibiting or reducing the proliferation of cancerous cells comprising an effective amount of a hedgehog inhibitor and an effective dose of radiation to inhibit or reduce the proliferation of the cells.
